(19) United States
(12) Patent Application Publication
(10) Pub. No.: US 2007/0249935 A1
(43) Pub. Date: Oct. 25, 2007

(54) System and method for automatically obtaining ultrasound image planes based on patient specific information

(75) Inventors: Harald Deschinger, Frankenmarkt (AT); Peter Falkensammer, Voecklabruck (AT)

Correspondence Address: DEAN D. SMALL;THE SMALL PATENT LAW GROUP LLP, 611 OLIVE STREET, SUITE 1611, ST. LOUIS, MO, 63101, US

(73) Assignee: GENERAL ELECTRIC COMPANY

(21) Appl. No.: 11/434,432

(22) Filed: May 15, 2006

Related U.S. Application Data
(60) Provisional application No. 60/793,908, filed on Apr. 20, 2006.

(51) Int. Cl. A61B 8/00

(52) U.S. Cl. 600/437

(57) ABSTRACT

A diagnostic ultrasound system is provided for automatically displaying multiple planes from a volume of interest. The system comprises a transducer for acquiring ultrasound data associated with a volume of interest having a target object therein. They system further comprises a user interface for designating a reference plane within the volume on interest. A processor module receives patient specific information representative of at least one of a shape and size of the target object and maps the reference plane and the ultrasound data into a 3D reference coordinate system. The processor module automatically calculates at least one plane of interest within the 3D reference coordinate system based on the reference plane and the patient specific information.

### RELATED APPLICATION

The present application relates to and claims priority from Provisional Application Ser. No. 60/793,908 filed Apr. 20, 2006 titled “SYSTEM AND METHOD FOR AUTOMATICALLY OBTAINING ULTRSOUND IMAGE PLANES BASED ON PATIENT SPECIFIC INFORMATION”, the complete subject matter of which is hereby expressly incorporated in its entirety.

### BACKGROUND OF THE INVENTION

Embodiments of the present invention relate generally to systems and methods for automatically obtaining ultrasound image planes of the volume of interest, and more specifically for automatic image plane calculation based upon patient specific information.

Ultrasound systems are used in a variety of applications and a by a variety of individuals with varied levels of skill. In many examinations, operators of the ultrasound system review selected combinations of ultrasound images in accordance with predetermined protocols. In order to obtain the desired combination of ultrasound images, the operator steps through a sequence of operations to identify and capture one or more desired image planes. At least one ultrasound system has been proposed, generally referred to in as automated multiplanar imaging that seeks to standardize acquisition and display of the desired image planes. In accordance with this recently proposed ultrasound system, a volumetric image is acquired in a standardized manner and a reference plane is identified. Based upon the reference plane, multiple image planes are automatically obtained from an acquired volume of ultrasound information without detailed intervention by the user to select each of the multiple image planes.

However, conventional ultrasound systems have experience certain limitations. The conventional automated multiplanar imaging process progresses independent of, and without consideration for, characteristics of the target object that render the target object unique and size and shape. Consequently, when a reference plane is identified, the multiple images that are automatically calculated may not be properly positioned within or relative to the target object if the size and shape of the target object differ from the standard.

A need remains for an improved method and system that affords automated multiplanar imaging, while remaining adaptable to different types, shapes and sizes of objects.

### BRIEF DESCRIPTION OF THE INVENTION

In accordance with an embodiment of the present invention, a diagnostic ultrasound system is provided for automatically displaying multiple planes from a volume of interest. The system comprises a transducer for acquiring ultrasound data associated with a volume of interest having a target object therein. They system further comprises a user interface for designating a reference plane within the volume on interest. A processor module receives patient specific information representative of at least one of a shape and size of the target object and maps the reference plane and the ultrasound data into a 3D reference coordinate system. The processor module automatically calculates at least one plane of interest within the 3D reference coordinate system based on the reference plane and the patient specific information.

For example, the volume of interest may constitute an organ of a fetus (e.g. the myocardium, the head, a limb, the liver, an organ and the like). The patient specific information may include geometric parameters (e.g. diameter, circumference, an organ type identifier in the like). Alternatively, or in addition, the patient specific information may include non-geometric parameters (e.g. age, weight, sex and the like). Optionally, the processor module may calculate a translation distance and a rotation distance from the reference plane to determine a position and orientation of the plane of interest within the 3D reference coordinate system, wherein the translation and rotation distances are based on an age of a patient.

### BRIEF DESCRIPTION OF THE DRAWINGS

FIG. 1 illustrates a block diagram of a diagnostic ultrasound system formed in accordance with an embodiment of the present invention.

FIG. 2 illustrates a table storing an association between patient specific information and automatic image planes to be generated in accordance with an embodiment of the present invention.

FIG. 3 represents a graphical representation of image planes that may be automatically calculated from a reference plane in accordance with an embodiment of the present invention.

FIG. 4 represents and other graphical representation of image planes that may be automatically calculated from a reference plane in accordance with an embodiment of the present invention.

FIG. 5 illustrates a processing sequence to obtain ultrasound image planes from a pre-acquired 3-D data set in accordance with an embodiment of the present invention.

FIG. 6 illustrates a processing sequence to obtain selected 2-D ultrasound image planes in accordance with an embodiment of the present invention.

FIG. 7 illustrates a processing sequence to obtain ultrasound image planes based upon measured anatomic structures in accordance with an embodiment of the present invention.

FIG. 8 illustrates a processing sequence to obtain ultrasound image planes a real-time continuously updated 3-D data set in accordance with an embodiment of the present invention.

### DETAILED DESCRIPTION OF THE INVENTION

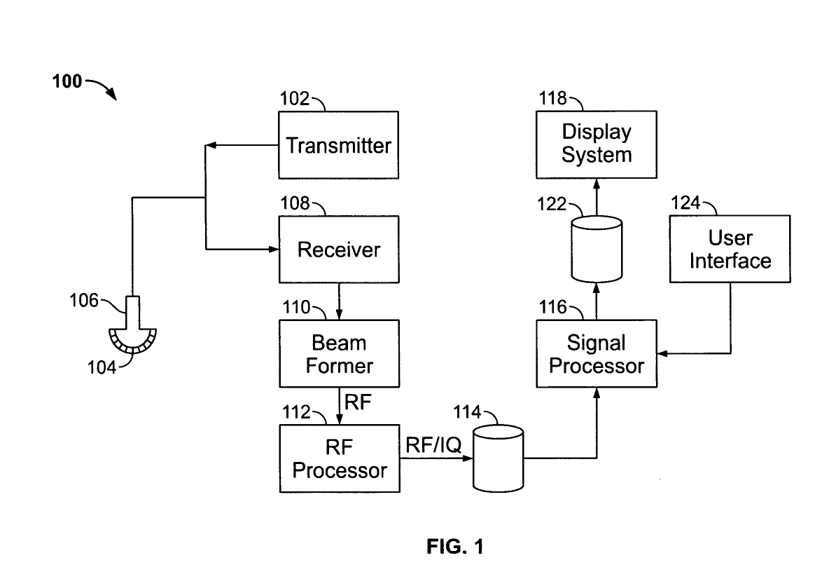

FIG. 1 illustrates a block diagram of an ultrasound system 100 formed in accordance with an embodiment of the present invention. The ultrasound system 100 includes a transmitter 102 which drives an array of elements 104 within a transducer 106 to emit pulsed ultrasonic signals into a body. A variety of geometries may be used. The ultrasonic signals are back-scattered from structures in the body, like blood cells or muscular tissue, to produce echoes which return to the elements 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to RF/IQ buffer 114 for temporary storage.

The ultrasound system 100 also includes a signal processor 116 to process the acquired ultrasound information (i.e., RF signal data or IQ data pairs) and prepare frames of ultrasound information for display on display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation. An image buffer 122 is included for storing processed frames of acquired ultrasound information that are not scheduled to be displayed immediately. The image buffer 122 may comprise any known data storage medium.

The signal processor 116 is connected to a user interface 124 that controls operation of the signal processor 116 as explained below in more detail. The display system 118 includes one or more monitors that present patient information, including diagnostic ultrasound images to the user for diagnosis and analysis.

The system 100 obtains volumetric data sets by various techniques (e.g., 3D scanning, real-time 3D imaging, volume scanning, 2D scanning with transducers having positioning sensors, freehand scanning using a Voxel correlation technique, 2D or matrix array transducers and the like). The transducer 106 is moved, such as along a linear or arcuate path, while scanning a region of interest (ROI). At each linear or arcuate position, the transducer 106 obtains scan planes that are stored in the memory 114.

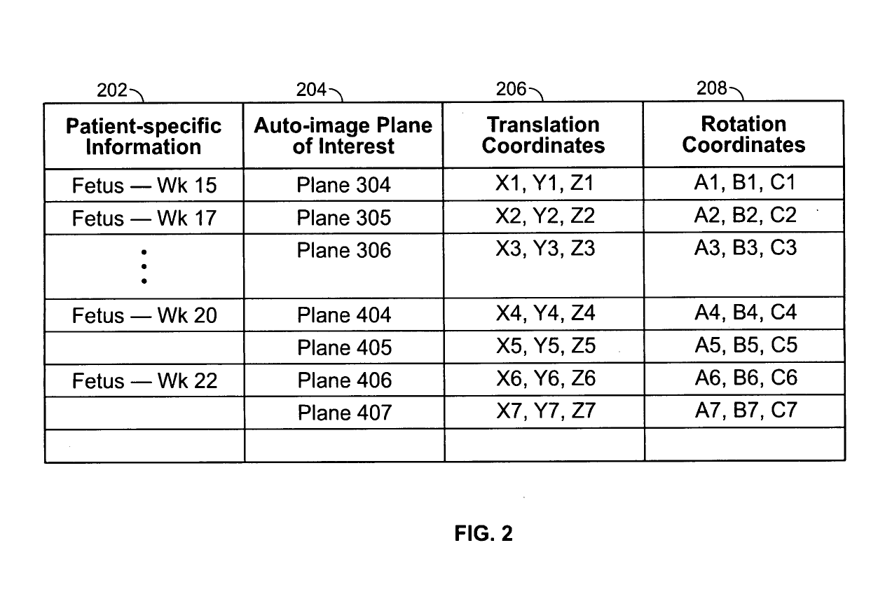

FIG. 2 illustrates a table 200 that stores the relation between patient specific information 202 and predetermined automatic image planes of interest 204. Each plane of interest 204 is associated in the table 200 with a series of translation and rotation coordinates 206 and 208, respectively. In the example of FIG. 2, the three-dimensional reference coordinate system is in Cartesian coordinates (e.g. XYZ). Thus, the translation coordinates 206 represent translation distances along the X, Y and Z axes. The rotation coordinates 208 represent rotation distances about the X, Y and Z axes. The translation and rotation coordinates 206, 208 extend from a reference plane.

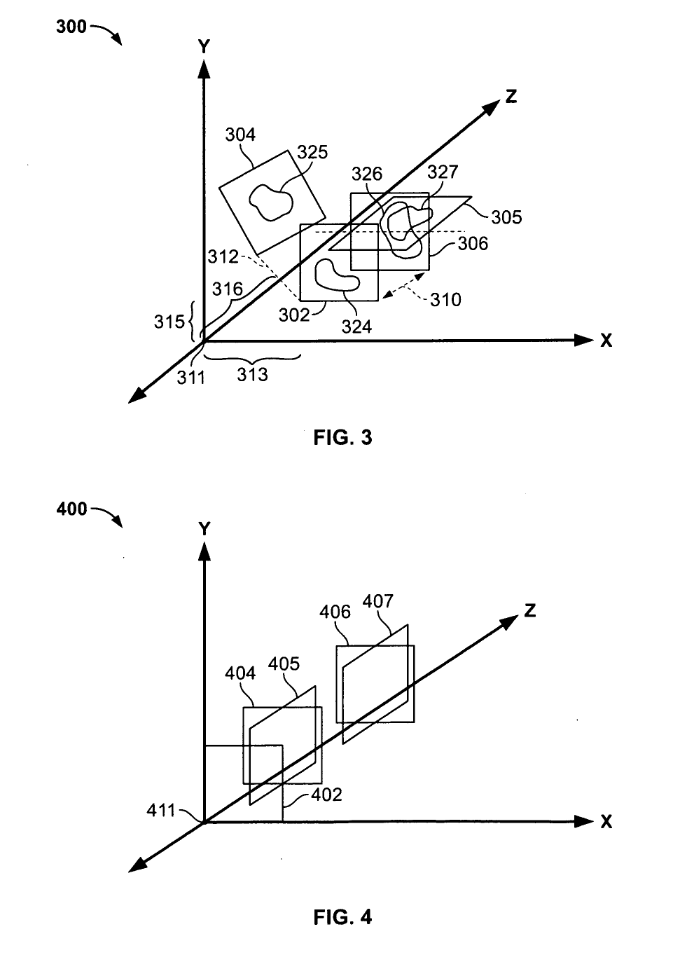

FIG. 3 represents a graphical representation of image planes that may be automatically calculated from a reference plane in accordance with an embodiment of the present invention. FIG. 3 illustrates a three-dimensional reference coordinate system 300, in which a reference plane 302 has been designated. The reference plane 302 may be acquired as a single two-dimensional image (e.g. B-mode image or otherwise). Alternatively, the reference plane 302 may be acquired as part of a three-dimensional scan of a volume of interest. For example, the reference plane may constitute a four chamber view of a fetal heart, the right ventricular outflow, the left ventricular outflow, the ductal arch, the aortic arch, venous connections, and the three vessel view. Once the reference plane 302 is adjusted and reoriented until the reference plane 302 contains a reference anatomy 324. Once the reference plane 302 is acquired, it is mapped into the 3-D reference coordinate system 300. In the example of FIG. 3, the reference plane 302 is located distances 313-316 from the origin 311 of the 3-D reference coordinate system 300 along the X, Y, and Z axes.

After acquiring the reference plane 302 and the fetal age, the processor module 116 automatically calculates additional image planes of interest based upon patient specific information, such as the age of a fetus. The patient specific information may constitute a geometric parameter, and nongeometric parameter or a combination thereof. The patient specific information may provide one-dimensional, two-dimensional or three-dimensional information regarding the target organ. Examples of geometric parameters are an identification of a type of organ, a diameter, a circumference, a length, an organ dimension and the like. The type of organ may be the heart, head, liver, arm, leg or other organ. Examples of non-geometric parameters are age, weight, sex and the like. For example, when examining a fetus that is in week 15 of gestation, a fetal organ or area of interest may be positioned, relative to the reference anatomy 324, at a position denoted by image 325. Once the processor module 116 receives the fetal age, processor module accesses the table 200 to obtain the translation coordinates X1, Y1, and Z1 and the rotation coordinates A1, B1, and C1. The position and orientation of the image plane 304 is determined from the translation and rotation coordinates.

Alternatively, when the fetus is in week 17, a fetal organ or area of interest may be positioned, relative to the reference anatomy 324, at a position denoted by images 326 and 327. After acquiring the reference plane 302 and the fetal age, the processor module 116 automatically calculates the positions and orientations of image planes 305 and 306. The image planes 305-306 of interest are located within the 3-D reference coordinate system 300, but are translated and rotated from the position of the reference plane 302 by predetermined distances.

Thus, the positions of each image plane 304-306 is defined relative to the reference plane 302 based upon the fetal age. For example, image plane 306 is translated in the Z direction by a distance 310 from the reference plane 302, while the image plane 304 is rotated about the Z axis by a predetermined arc in degrees 312. The image plane 305 is both translated and rotated about multiple axes from the reference plane 302.

FIG. 4 represents another graphical representation of image planes that may be automatically calculated from a reference plane in accordance with an embodiment of the present invention. In FIG. 4, a three-dimensional reference coordinate system 400 is illustrated in Cartesian coordinates. Optionally, the coordinate reference system may be defined in polar accordance. Optionally, the reference plane 402 may be mapped to the origin 411 of the reference coordinate system 400. In the example of FIG. 4, image planes 404 and 405 are automatically calculated based upon the reference plane 402 when the fetus is 20 weeks old, while image planes 406-407 are automatically calculated based upon the reference plane 402 when the fetus is 22 weeks old. The image planes 406-407 are spaced further from the reference plane 402, along the Z direction, to account for the increased length of the organ of interest.

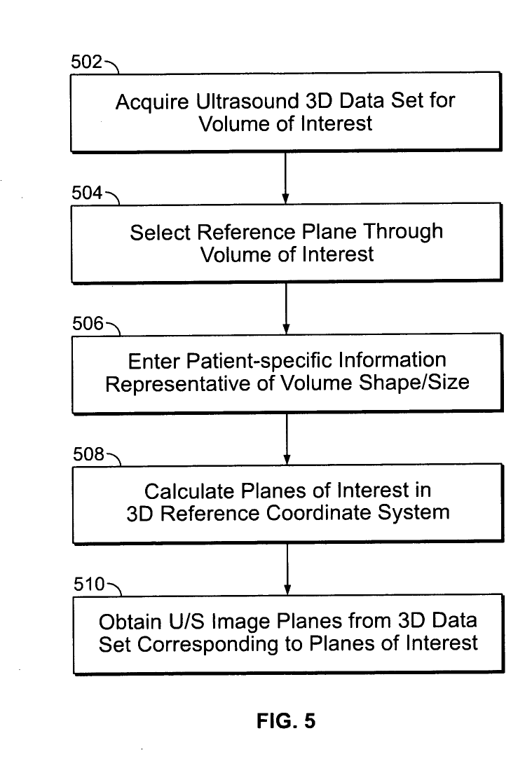

FIG. 5 illustrates a processing sequence to obtain ultrasound image planes from a pre-acquired 3-D data set in accordance with an embodiment of the present invention. Beginning at 502, a 3-D data set of ultrasound data is acquired for a volume of interest. At 504, the user selects a reference plane from the volume of interest. Once the user selects the reference plane, the reference plane may be mapped into a three-dimensional reference coordinate system. At 506, patient specific information is entered that represents the shape and/or size of the organ of interest within the volume of interest. For example, the patient specific information may be manually entered by the user (e.g. entering the age of a fetus). Alternatively, the patient specific information may be automatically calculated from other anatomic characteristics or structures within the reference plane. As a further option, the patient specific information may be obtained by accessing medical records previously saved and updated for the patient under examination. For example, the age of a fetus may be automatically calculated based upon the social security number or other unique ID of the patient by accessing the patient's medical records previously entered and updated in accordance with a pregnancy.

At 508, one or more image planes of interest are calculated within the three-dimensional reference coordinate system. At 510, ultrasound images, associated with the automatically calculated image planes, are obtained from the 3-D data set and presented as ultrasound images to a user in a desired format.

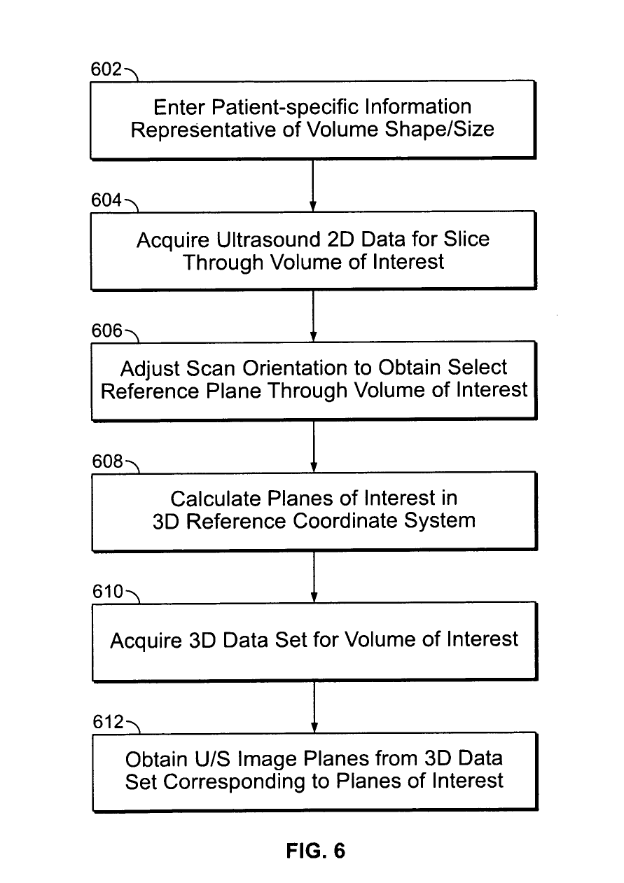

FIG. 6 illustrates a processing sequence to obtain select 2-D ultrasound image planes in accordance with an embodiment of the present invention. At 602, patient specific information is entered that represents the shape or size of the organ of interest within the volume of interest. At 604, a two-dimensional ultrasound slice or scan is acquired from within a volume of interest. At 604, the system need not yet perform a complete three-dimensional volumetric scan. Instead, at 604, a single slice or planar scan may be acquired. At 606, the user is afforded the ability to adjust the orientation and position of the probe in order to acquire a desired reference plane through a volume of interest. At 608, one or more image planes are calculated within a 3-D reference coordinate system based upon the select reference plane and the patient specific information. At 610, one or more select two-dimensional image planes are acquired from within the volume of interest. The acquired select 2-D image planes correspond to the image planes of interest calculated at 608. Optionally, the entire volume of interest need not be scanned, but instead the system need only acquire ultrasound information for the select 2-D image planes of interest. At 612, ultrasound images are displayed for the image planes of interest.

Optionally, any embodiment of FIG. 6, the ultrasound images associated with the select image planes may be continuously updated in real-time at a frame rate sufficiently high, relative to a fetal heart rate, to provide meaningful motion information.

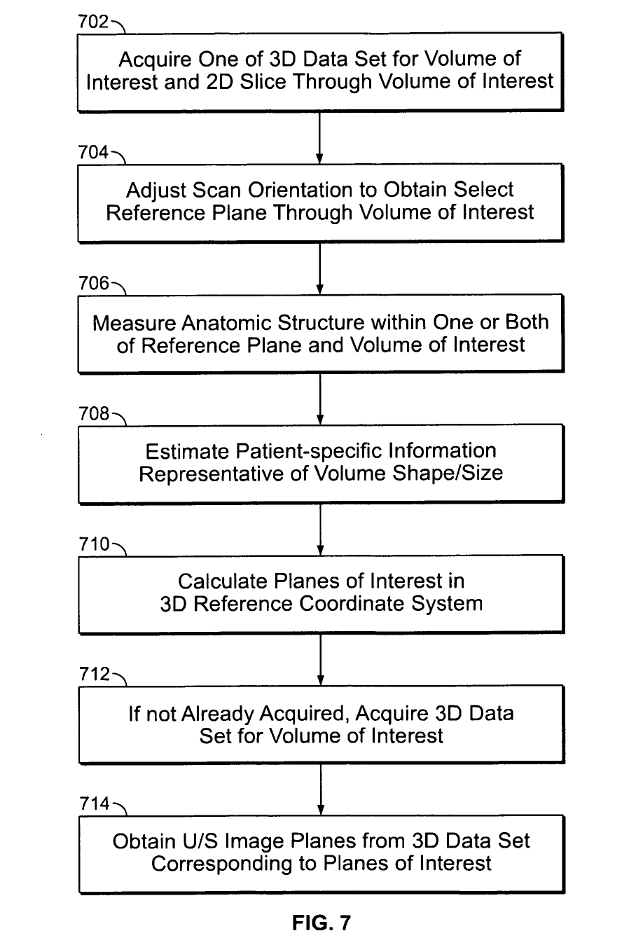

FIG. 7 illustrates a processing sequence to obtain ultrasound image planes based upon measured anatomic structures in accordance with an embodiment of the present invention. Beginning at seven are two, the system acquires one of a 3-D data set for the volume of interest or one or more two-dimensional slices through the volume of interest. At 704, the user adjusts the scan orientation to obtain a select reference plane through the volume of interest. At 706, a measurement is obtained for an anatomic structure within one or both of the reference planes and the volume of interest. For example, the anatomic structure may represent a select bone within a fetus. By measuring the length of the select bone, the fetal age may be automatically determined.

At 708, the patient specific information is estimated representative of the shape or size of the volume. At 710, the image planes of interest are calculated from the 3-D reference coordinate system and at 712 a 3-D data set is acquired (unless already completed). At 714, one or more ultrasound images are displayed corresponding to the image planes of interest.

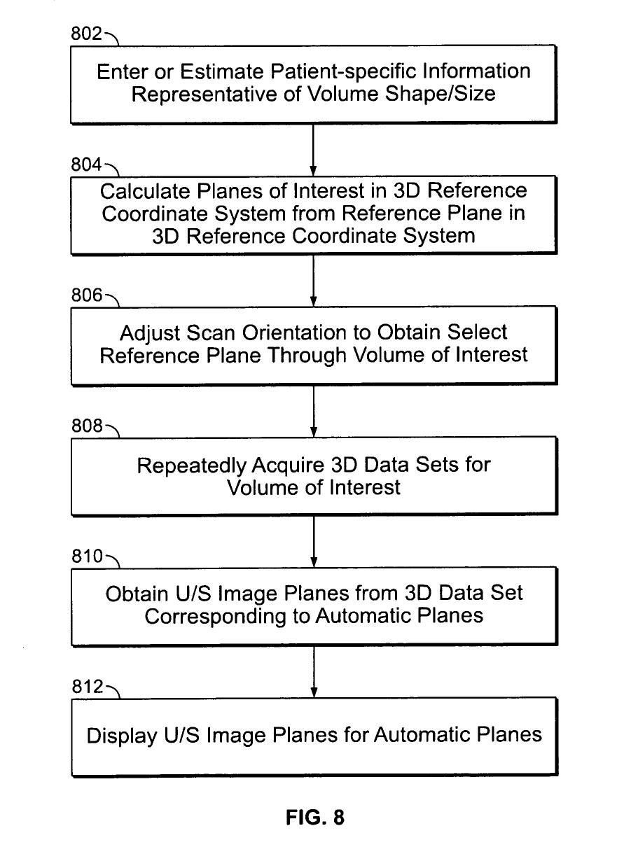

FIG. 8 illustrates a processing sequence to obtain ultrasound image planes in a real-time continuously updated 3-D data set in accordance with an embodiment of the present invention. At 802, patient specific information is estimated or entered. The patient specific information is representative of the shape or size of the volume. At 804, image planes of interest are calculated in the 3-D reference coordinate system. In the example of FIG. 8, a reference plane has not yet been calculated at 804. Instead, at 804, the image planes are calculated relative to the origin of a predetermined 3-D reference coordinate system. The image planes are projected into the predetermined 3D reference coordinate system based upon the assumption that the reference coordinate system and the subsequently acquired volumetric data set will be mapped in a known manner within, and relative to the origin, of the 3-D reference coordinate system.

At 806, the probe is positioned to obtain a select reference plane through the volume of interest. At 808, a 3-D data set of volumetric ultrasound data is acquired. The volumetric data set is mapped into the 3-D reference coordinate system such that the reference plane is positioned at a known location and orientation relative to the origin of the 3-D reference court system. At 810, ultrasound images are obtained for the image planes calculated at 804. At 812, the ultrasound images are displayed.

It is understood that the above methods and systems may be utilized in connection with a variety of patient types, diagnoses, organs and the like. For example, the organ may be the heart, head, liver, arm, leg and the like.

While the invention has been described in terms of various specific embodiments, those skilled in the art will recognize that the invention can be practiced with modification within the spirit and scope of the claims.

What is claimed is:

1. A diagnostic ultrasound system for automatically displaying multiple planes from a volume of interest, the system comprising:
a transducer acquiring ultrasound data associated with a volume of interest that includes a target object;
an user interface for designating a reference plane within the volume on interest; and
a processor module receiving patient specific information representative of at least one of a shape and size of the target object, the processor module mapping the reference plane and the ultrasound data into a 3D reference coordinate system, the processor module automatically calculating at least one plane of interest within the 3D reference coordinate system based on the reference plane and the patient specific information.

2. The system of claim 1, wherein the patient specific information constitutes a geometric parameter that includes at least one of an identification of a type of organ, a diameter, a circumference, a length, and organ dimension.

3. The system of claim 1, wherein the processor module calculates a translation distance and a rotation distance from the reference plane to determine a position and orientation of the plane of interest within the 3D reference coordinate system, wherein the translation and rotation distance is based on an age of a patient.

4. The system of claim 1, wherein the patient specific information constitutes a non-geometric parameter including at least one of age, weight and sex.

5. The system of claim 1, further comprising memory storing a 3-D data set of ultrasound data associated with the volume of interest, the reference plane representing a user defined plane within the volume of interest, wherein the 3-D data set is acquired before the plane of interest is calculated.

6. The system of claim 1, further comprising memory storing and repeatedly updating 3-D data set of ultrasound data associated with the volume of interest, the reference plane representing a user defined plane within the volume of interest, wherein the 3-D data set is continuously updated before and after the plane of interest is calculated.

7. The system of claim 1, further comprising memory storing a table including predefined sets of translation and rotation values in connection with corresponding planes of interest, each set of translation and rotation values being associated with the patient specific information.

8. The system of claim 1, wherein the patient specific information includes an age of a fetus and the processor module calculates a relation between the plane of interest and the reference plane based on a plurality of preceding fetal studies of other patients.

9. A method for automatically displaying multiple ultrasound planes from a volume of interest, the method comprising:
acquiring ultrasound data associated with a volume of interest that includes a target object;
designating a reference plane within the volume on interest;
receiving patient specific information representative of at least one of a shape and size of the target object;
mapping the reference plane and the ultrasound data into a 3D reference coordinate system; and
automatically calculating at least one plane of interest within the 3D reference coordinate system based on the reference plane and the patient specific information.

10. The method of claim 9, wherein the patient specific information constitutes a geometric parameter that includes at least one of an identification of a type of organ, a diameter, a circumference, a length, and organ dimension.

11. The method of claim 9, further comprising calculating a translation distance and a rotation distance from the reference plane to determine a position and orientation of the plane of interest within the 3D reference coordinate system, wherein the translation and rotation distance is based on an age of a patient.

12. The method of claim 9, wherein the patient specific information constitutes a non-geometric parameter including at least one of age, weight and sex.

13. The method of claim 9, further comprising memory storing a 3-D data set of ultrasound data associated with the volume of interest, the reference plane representing a user defined plane within the volume of interest, wherein the 3-D data set is acquired before the plane of interest is calculated.

14. The method of claim 9, wherein the volume of interest includes a fetus, the method further comprising measuring an anatomic structure within the reference plane and based upon the measurement of the anatomic structure, determining an age of the fetus.

15. The method of claim 9, wherein a 3-D data set of ultrasound data is obtained after the plane of interest has been calculated.
